(19) United States
(12) Patent Application Publication
(10) Pub. No.: US 2017/0304576 A1
(43) Pub. Date: Oct. 26, 2017

(54) STRAP MEMBER FOR A PATIENT INTERFACE

(71) Applicant: KONINKLIJKE PHILIPS N.V., EINDHOVEN (NL)

(72) Inventors: MATTHEW JOHN LAWRENSON, BUSSIGNY-PRES-DE-LAUSANNE (CH); JULIAN CHARLES NOLAN, PULLY (CH)

(21) Appl. No.: 15/526,042

(22) PCT Filed: Oct. 27, 2015
(86) PCT No.: PCT/EP2015/074833

§ 371 Date: May 11, 2017

(30) Foreign Application Priority Data
Nov. 19, 2014 (EP) 14193918.1

(51) Int. Cl. A61M 16/06; A61M 16/16; A61M 16/20

(52) U.S. Cl. CPC A61M 16/0683; A61M 16/202; A61M 16/161; A61M 2205/362; A61M 2205/3344; A61M 2205/3303; A61M 2210/04; A61M 2230/50; A61M 2205/3606

(57) ABSTRACT

The present invention relates to a strap member (28a-c) for use in a patient interface (10) for supplying a pressurized flow of breathable gas to an airway of a patient (12), wherein the strap member (28a-c) is configured to secure the patient interface (10) to the head of the patient (12) and comprises: —a strap portion (38) having (i) a first side (40) that is configured to contact the face of the patient (12) during use, (ii) an internal chamber forming a cavity (30), and (iii) a plurality of openings (44) which are fluidly connected to the cavity (30) and arranged at the first side (40); and —a connector (32) for fluidly connecting the cavity (30) to the pressurized flow of breathable gas supplied to the airway of the patient (12).

### FIELD OF THE INVENTION

The present invention relates to a strap member of a headgear for use in a patient interface for supplying a pressurized flow of breathable gas to an airway of a patient. The present invention particularly relates to a strap member that reduces a moisture build up and/or warm-up between the headgear and the skin of the patient. Further, the present invention relates to a patient interface and to a pressure support system including such a strap member.

### BACKGROUND OF THE INVENTION

Patient interfaces, such as masks in pressure support systems, are used for delivering gas to a user. Such gases like air, cleaned air, oxygen, or any modification thereof are submitted to the user (also referred to as patient) via the patient interface in a pressurized or unpressurized way.

For several chronic disorders and diseases the usage of such a patient interface is necessary or at least advisable.

One example of such a disease is obstructive sleep apnea or obstructive sleep apnea syndrome (OSA). OSA is usually caused by an obstruction of the upper airway. It is characterized by repetitive pauses in breathing during sleep and is usually associated with a reduction in blood oxygen saturation. These pauses in breathing, called apneas, typically last 20 to 40 seconds. The obstruction of the upper airway is usually caused by reduced muscle tonus of the body that occurs during sleep. The human airway is composed of walls of soft tissue which can collapse and thereby obstruct breathing during sleep. Tongue tissue moves towards the back of the throat during sleep and thereby blocks the air passages. OSA is therefore commonly accompanied with snoring.

Different invasive and non-invasive treatments for OSA are known. One of the most powerful non-invasive treatments is the usage of Continuous Positive Airway Pressure (CPAP) or Bi-Positive Airway Pressure (BiPAP) in which a patient interface, e.g. a face mask, is attached to a tube and a machine that blows pressurized gas, preferably air, into the patient interface and through the airway of the patient in order to keep it open. Positive air pressure is thus provided to a patient through a hose connected to a patient interface or respiratory interface, such as a face mask, that is worn by the patient regularly at night. The afore-mentioned long-term use of the patient interface is the result, since the wearing of the patient interface usually takes place during the sleeping time of the patient.

Examples for patient interfaces are:
nasal masks, which fit over the nose and deliver gas through the nasal passages,
oral masks, which fit over the mouth and deliver gas through the mouth,
full face masks, which fit over both, the nose and the mouth, and deliver gas to both, and
nasal pillows, which are regarded as masks as well within the scope of the present invention and which consist of small nasal inserts that deliver the gas directly to the nasal passages.

In order to guarantee a reliable operation of the device, the patient interface needs to closely fit on the patient's face to provide an air-tight seal at the mask-to-face interface. The patient interface is worn using a headgear with straps that go around the back of the patient's head. These straps are often made of an elastic textile material. The patient interface or mask in practice usually comprises a soft cushion that is used as mask-to-patient interface, i.e. that contacts the face of the patient when the mask is worn, as well as it usually comprises a so-called mask shell building a rigid or semi-rigid holding structure for holding the cushion in place and for supplying mechanical stability to the patient interface.

The cushion usually comprises one or more pads made of gel or silicone or any other soft material in order to increase the patient comfort and guarantee a soft feeling on the patient's face. The latter-mentioned mask shell is usually made of polycarbonate and normally further comprises a hose interface that is adapted for connecting the air supplying hose to the mask. Depending on the type of the mask, it may also comprise a mechanism with an additional cushion support on the forehead (also denoted as forehead support) to balance the forces put by the mask around the airway entry features of the human face.

A general problem which occurs with regard to the headgear is the fact that the area between the patient's skin underneath the headgear straps may become warm. This may also lead to a moisture build-up underneath the headgear straps. Especially after a long-term usage of the patient interface this may cause skin irritations on the patient's skin which leads to discomfort. It goes without saying that the patient adherence to wearing such patient interfaces is thereby reduced.

US 2013/0263859 A1 discloses a strap member of a patient interface which includes a breathability enhancing element. This breathability enhancing element may include a number of micro bumps arranged on the side of the headgear strap which contacts the patient's face during use. The breathability enhancing element may also comprise a number of apertures which extend through the headgear strap such that the area underneath the headgear strap is provided with ambient air.

While the measures proposed in US 2013/0263859 A1 have shown to be effective, there is still room for improvement.

WO 2011/110962 A1 discloses patient interface device that includes a patient sealing element and a tubing assembly fluidly coupled to the patient sealing element for delivering a breathing gas to the patient sealing element. The tubing assembly is adapted to be worn on a head of a user and includes at least one rigid or semi-rigid straight segment fluidly coupled to at least one flexible bellows segment. The tubing assembly may be provided within a headgear component used to attach the patient interface device to the patient's head.

EP 1 645 258 A1 discloses an overlay for a patient support. The overlay is coupled to an air supply and includes an air permeable three-dimensional fiber network.

WO 2013/156960 A1 discloses a patient interface with at least one elongated support member structured to contact a user. The support member has at least one conduit portion and at least one porous portion. The conduit portion is structured to allow the passage of gas therethrough. The conduit portion is structured to be in fluid communication with a pressure generating system and with the porous portion. The porous portion is structured to allow for axial passage of gas therethrough and for radial exhaust of gas therefrom.

### SUMMARY OF THE INVENTION

It is an object of the present invention to provide an improved strap member for a headgear strap of a patient interface that solves the above-mentioned problems. It is particularly an object of the present invention to provide a strap member of a headgear of a patient interface that enhances the patient's comfort and reduces or even eliminates an uncomfortable warm-up or moisture build-up underneath the headgear strap during usage. It is furthermore an object of the present invention to provide a patient interface and a pressure support system comprising a headgear with such a strap member.

In a first aspect of the present invention a strap member for use in a patient interface for supplying a pressurized flow of breathable gas to an airway of a patient is presented. The strap member is configured to secure the patient interface to the head of the patient and comprises:
a strap portion having (i) a first side that is configured to contact the face of the patient during use, (ii) an internal chamber forming a cavity, and (iii) a plurality of openings which are fluidly connected to the cavity and arranged at the first side;
a flow restriction element for restricting and/or regulating an inflow of the pressurized flow of breathable gas into the cavity; and
a connector for fluidly connecting the cavity to the pressurized flow of breathable gas supplied to the airway of the patient during use of the patient interface.

In a further aspect of the present invention a patient interface for supplying a pressurized flow of breathable gas to an airway of a patient is presented, comprising:
a sealing arrangement for providing a substantially air-tight sealing connection with an airway opening of the patient;
an inlet for receiving the pressurized flow of breathable gas which is fluidly connected to an interior of the sealing arrangement; and
a headgear including a strap member as claimed in claim 1;

wherein the interior of the sealing arrangement is fluidly connected to the cavity of the strap member by means of the connector.

In a still further aspect of the present invention a pressure support system is presented which comprises a pressure generator for generating a pressurized flow of breathable gas and a patient interface of the above-mentioned type.

Preferred embodiments of the invention are defined in the dependent claims. It shall be understood that the claimed patient interface and the claimed pressure support system have similar and/or identical preferred embodiments as the claimed strap member and as defined in the dependent claims.

One of the central ideas of the present invention is to use at least a part of the pressurized gas flow that is supplied to the airway of the patient also as a source of cooling gas in order to cool and/or dry the patient's skin underneath a strap member of the headgear. The strap member thereto comprises an internal chamber forming a cavity which is fluidly connected to the pressurized flow of breathable gas by means of a connector. The connector may, for example, be connected to the sealing arrangement of the patient interface which provides a substantially air-tight sealing connection with an airway opening of the patient. In this way, the pressurized flow of breathable gas is not only guided towards the airway openings of the patient, but also into the interior of the headgear strap. The headgear strap also comprises a plurality of openings which are fluidly connected to the cavity, i.e. to the interior of the headgear strap, and arranged at the side of the headgear strap which contacts the patient's face during use. A part of the pressurized gas flow may thus exit the headgear strap through these openings and will be guided to the skin areas which are covered by the headgear strap.

One and the same gas flow is therefore not only used for delivery to the airway openings of the patient, but also used to cool and dry the skin areas of the patient underneath the headgear strap. A formation of moisture, sweat and skin irritations, such as red marks, may thus be prevented.

The first side of the strap portion which contacts the patient's face preferably comprises a perforated material which comprises the plurality of openings. A second side of the strap portion opposite the first side preferably comprises a substantially air-tight material. This ensures that the gas flow exits the headgear strap only in the direction towards the patient's skin. This also reduces the pressure loss within the headgear and the sealing arrangement (mask).

The flow restriction element provides the advantage that the gas pressure within the strap member of the headgear is lower than the gas pressure occurring in the sealing arrangement, i.e. in the mask. The gas pressure occurring within the cavity of the strap member may thus be at least partly regulated. It is particularly advantageous that the gas flow for keeping the airways of the patient open is stronger than the gas flow for cooling/drying the skin areas underneath the strap member of the headgear.

The restriction element itself may be realized in many ways. One may either use a material constriction at the interface between the sealing arrangement (the mask) and the strap member of the headgear in order to restrict the inflow of the gas into the cavity. Alternatively, a valve may be used as a flow restriction element.

According to a preferred embodiment, the flow restriction element comprises a valve. This valve may be closed or at least partly closed in some instances to restrict the inflow of the gas into the cavity. The valve is thereto preferably arranged at or near the connector, i.e. at the interface between the sealing arrangement and the strap member. The valve allows a regulation of the gas flow exiting the plurality of openings towards the skin of the user underneath the strap member.

According to an embodiment, the strap member may comprise a pressure sensor for measuring a pressure within the cavity, and a controller for controlling the valve, wherein the controller is configured to control the valve depending on the pressure measured by the pressure sensor.

The controller maybe configured to close the valve if the pressure within the cavity is above a predefined maximum threshold value. The controller may be configured to open the valve if the pressure within the cavity is below a predefined minimum threshold value. In this way the gas pressure within the cavity may be maintained within a certain pressure range that is suitable for cooling/drying the skin areas underneath the strap member of the headgear.

According to a further embodiment, the strap member further comprises a controller for controlling the valve, wherein the controller is configured to control the valve in a time-dependent manner, in particular to open the valve during predetermined time intervals.

The valve may, for example, be opened every half hour for 30 seconds. Other time intervals are of course possible as well. It is also possible to open the valve at predefined times of the day or the night. The valve may, for example, only be opened every night between 1 a.m. and 5 a.m. when it is expected that the patient is in deep sleep. During these time periods, the controller may either open up the valve permanently or in predefined time intervals. The patient is thus less likely to be disturbed by the gas flow that is used for cooling/drying his/her skin underneath the strap member.

According to a further embodiment, the strap member may further include a humidity sensor and/or a temperature sensor for sensing a humidity/temperature at or near the first side of the strap portion, and a controller for controlling the valve, wherein the controller is configured to control the valve depending on the humidity/temperature measured by the humidity sensor and/or the temperature sensor.

The gas inflow into the cavity is in this case modulated in accordance with a need for cooling/drying. The valve is only opened if the humidity/temperature sensor detects that cooling/drying underneath the strap member is effectively needed. This may further reduce the risk of unnecessarily disturbing the patient by the gas flow within the strap member.

According to a still further embodiment, the strap member may comprise a vital sign sensor for measuring a vital sign (e.g. pulse, blood pressure, blood oxygenation) of the patient, and a controller for controlling the valve, wherein the controller is configured to control the valve depending on the vital sign measured by the vital sign sensor.

In this case it is, for example, possible to open the valve only if it is detected that the pulse of the patient is below a certain threshold value that is indicative that the patient is asleep. The valve may thus be controlled depending on a sleep state of the patient.

According to a further embodiment, the connector for connecting the cavity of the strap member to the pressurized flow of gas comprises an inlet opening and a conical tube that is inserted into the inlet opening.

The conical tube may, for example, be inserted into the inlet opening in such a way that the part with the larger diameter of the conical tube is arranged within the sealing arrangement (the mask) and the part with the smaller diameter of the conical tube is arranged within the cavity of the strap member. Such an arrangement ensures an effective and air-tight sealing. Since the pressure occurring within the mask is typically higher than the pressure occurring within the strap member of the headgear, the conical tube will be automatically pressed into the cavity of the strap member.

As it was mentioned in the beginning, the present invention does not only relate to the above-mentioned strap member, but also to a patient interface comprising a headgear with such a strap member, as well as to a pressure support system comprising a pressure generator and the patient interface.

In case of the above-mentioned embodiment, in which the strap member includes a valve for regulating an inflow of the pressurized flow of breathable gas into the cavity, the pressure support system may comprise a controller that is configured to control the pressure generator to adapt the pressurized flow of breathable gas depending on a state of the valve, i.e. if the valve is open or closed. If the valve is closed, the controller may adapt the pressure generator to decrease the pressurized flow of breathable gas. If the valve is open, the controller may adapt the pressure generator to increase the pressurized flow of breathable gas. The controller for regulating the pressure generator may be the same controller as the controller for regulating the valve. However, it may also be an additional controller.

According to a further embodiment, the pressure support system may comprise a controller that is configured to control the pressure generator to adapt the pressurized flow of breathable gas depending on the pressure within the cavity of the strap member which may be measured, as explained above, by means of a pressure sensor that is arranged within the cavity. In this way a fairly constant pressure over time may be maintained within the cavity and the sealing arrangement of the patient interface.

In a second aspect of the present invention a strap member for use in a patient interface for supplying a pressurized flow of breathable gas to an airway of a patient is presented, wherein the strap member is configured to secure the patient interface to the head of the patient and comprises:
a strap portion having (i) a first side that is configured to contact the face of the patient during use, (ii) an internal chamber forming a cavity, and (iii) a plurality of openings which are fluidly connected to the cavity and arranged at the first side;
an actuator for regulating an outflow of the pressurized flow of breathable gas through at least one of the plurality of openings out of the cavity by changing at least one of a size, a shape, a location, and a geometry of the at least one of the plurality of openings; and
a connector for fluidly connecting the cavity to the pressurized flow of breathable gas supplied to the airway of the patient during use of the patient interface.

In a further aspect of the present invention a patient interface for supplying a pressurized flow of breathable gas to an airway of a patient is presented, comprising:
a sealing arrangement for providing a substantially air-tight sealing connection with an airway opening of the patient;
an inlet for receiving the pressurized flow of breathable gas which is fluidly connected to an interior of the sealing arrangement; and
a headgear including a strap member as claimed in claim 15;

wherein the interior of the sealing arrangement is fluidly connected to the cavity of the strap member by means of the connector.

In a still further aspect of the present invention a pressure support system is presented which comprises a pressure generator for generating a pressurized flow of breathable gas and a patient interface of the afore-mentioned type.

The difference between the strap member according to the second aspect of the present invention and the strap member according to the first aspect of the present invention is the implentation of an actuator for regulating the outflow out of the cavity instead of the implementation of a flow restriction element for restricting and/or regulating an inflow into the cavity. The effect of both measures is however similar, namely the possibility to regulate the gas pressure occurring within the cavity of the strap member as well as the possibility to regulate the gas flow for cooling/drying the skin areas underneath the strap member of the headgear. This increases the patient comfort.

The actuator regulates the outflow of the pressurized flow of breathable gas through at least one of the plurality of openings out of the cavity by changing at least one of a size, a shape, a location, and a geometry of the at least one of the plurality of openings. Such an actuator is preferably implemented as an electrically controllable actuator, most preferably as an electromechanical actuator.

The strap member according to the second aspect of the present invention preferably comprises, similar as the strap member according to the first aspect of the present invention, a second side of the strap portion opposite the first side, wherein said second side comprises a substantially air-tight material.

According to an embodiment, the actuator is arranged at or near the at least one of the plurality of openings.

According to a preferred embodiment, not only one of the plurality of openings may be changed in size, shape, location and/or geometry by means of the said actuator, but each of the plurality of openings may be changed in size, shape, location and/or geometry by means of the said actuator. The strap member may thereto either comprise (i) one common actuator which controls the size, shape, location and/or geometry of all openings or (ii) a plurality of actuators, wherein each of the plurality of actuators controls the size, shape, location and/or geometry of one of the plurality of openings.

The way the at least one actuator is controlled may be the same or similar as the above-mentioned alternatives for controlling the valve for regulating the inflow into the cavity. The explanations given above with respect to the different embodiments of controlling the valve for regulating the inflow into the cavity thus apply, mutatis mutandis, also for the following embodiments for controlling the actuator for regulating the outflow out of the cavity.

According to an embodiment, the strap member further comprises a pressure sensor for measuring a pressure within the cavity, and a controller for controlling the actuator, wherein the controller is configured to control the actuator depending on the pressure measured by the pressure sensor.

According to an embodiment, the strap member further comprises a controller for controlling the actuator, wherein the controller is configured to control the actuator to change the outflow of the pressurized flow of breathable gas through the at least one of the plurality of openings during predetermined time intervals.

According to an embodiment, the strap member further comprises a humidity sensor for sensing a humidity at or near the first side of the strap portion, and a controller for controlling the actuator, wherein the controller is configured to control the actuator to change the outflow of the pressurized flow of breathable gas through the at least one of the plurality of openings depending on the humidity measured by the humidity sensor.

According to an embodiment, the strap member further comprises a temperature sensor for sensing a temperature at or near the first side of the strap portion, and a controller for controlling the actuator, wherein the controller is configured to control the actuator to change the outflow of the pressurized flow of breathable gas through the at least one of the plurality of openings depending on the temperature measured by the temperature sensor.

### BRIEF DESCRIPTION OF THE DRAWINGS

These and other aspects of the invention will be apparent from and elucidated with reference to the embodiment(s) described hereinafter. In the following drawings

FIG. 1 shows a schematic side view of a patient wearing a patient interface according to an embodiment of the present invention;

FIG. 2 shows a detail of FIG. 1, in particular a connection between the sealing arrangement of the patient interface and the headgear;

FIG. 3 shows a schematic cross-section of a strap member according to a first embodiment of the present invention;

FIG. 4 shows a schematic cross-section of the strap member according to a second embodiment of the present invention;

FIG. 5 shows a schematic cross-section of the strap member according to a third embodiment of the present invention;

FIG. 6 shows a schematic cross-section of the strap member according to a fourth embodiment of the present invention;

FIG. 7 shows a schematic cross-section of the strap member according to a fifth embodiment of the present invention;

FIG. 8 shows a schematic block diagram illustrating the connections between possible components of the strap member according to the second and third embodiment of the present invention; and

FIG. 9 shows a schematic block diagram illustrating the connections between possible components of the strap member according to the fourth and fifth embodiment of the present invention.

### DETAILED DESCRIPTION OF THE INVENTION

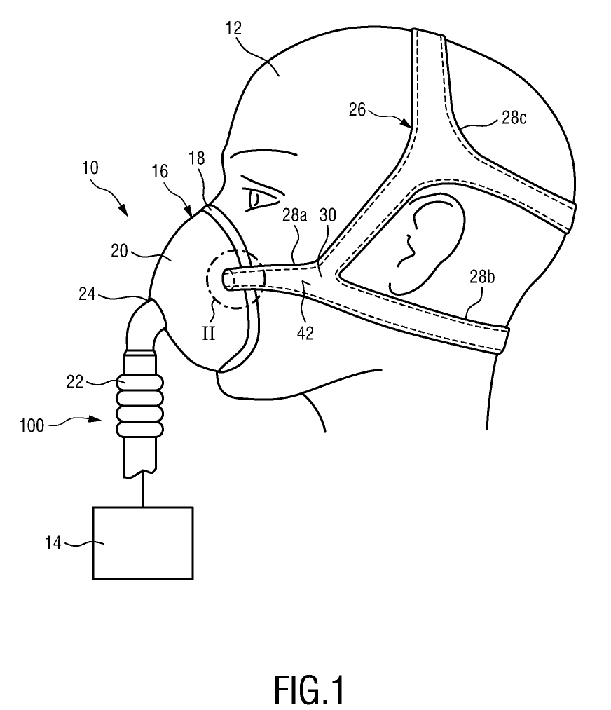

FIG. 1 shows an exemplary embodiment of a patient interface for supplying a pressurized flow of breathable gas to an airway of a patient. The patient interface is therein in its entirety denoted by reference numeral 10.

In this embodiment the patient interface 10 is designed as a full-face mask covering the mouth and the nose of a patient 12. It shall be noted that the patient interface 10 may alternatively be designed as a nose mask, a mouth mask or as a total face mask without leaving the scope of the present invention.

The patient interface 10 is part of a pressure support system that is indicated by reference numeral 100. The pressure support system 100 comprises, besides the patient interface 10, a pressure generator 14 for generating the pressurized flow of breathable gas. This pressure generator 14 is usually arranged remote from the patient interface 10, e.g. on a desk beside the bed of the patient 12. The pressure generator 14 usually comprises a ventilator or compressor for generating the pressurized flow of breathable gas and a controller for controlling the ventilator/compressor according to a predefined pressure cycle. The pressure generator 14 may either be configured to provide a constant flow rate (e.g. CPAP) or a flow rate that is varying over time (e.g. BiPAP).

According to the embodiment shown in FIG. 1, the patient interface 10 comprises a sealing arrangement 16 for providing a substantially air-tight sealing connection with an airway of the patient 12. In the shown example, this sealing arrangement 16 surrounds the nose and the mouth of the patient 12.

The sealing arrangement 16 comprises a cushion element 18 and a mask shell 20. The cushion element 18 is designed to contact the face of the patient 12 and to provide an air-tight seal at the interface between the patient's face and the patient interface 10. The cushion element 18 usually comprises a soft material, like silicon or any other rubber or suitable elastic material. The mask shell 20 provides a flexible, semi-rigid or rigid support structure for holding the cushion element 18. The mask shell 20 is usually connected to the backside of the cushion element 18, wherein the backside is meant to denote the side of the cushion element 18 opposite the side of the cushion element 18 contacting the patient's face during use. The mask shell 20 may be either releasably or fixedly connected to the cushion element 18. The cushion element 18 and the mask shell 20 together form a cavity for receiving the mouth and/or the nose of the patient 12 (in this case the mouth and the nose of the patient 12). It shall be noted that the cushion element 18 and the mask shell 20 may be also formed as one integral piece.

On the opposite side directing away from the patient's face, the mask shell 20 is connected to a hose 22 via which the pressurized flow of breathable gas is transmitted from the pressure generator 14 to the patient interface 10. The hose 22 is connected to an inlet 24 formed within the mask shell 20, such that the inlet 24 fluidly connects the interior of the sealing arrangement 16 to the hose 22 and the pressure generator 14.

The sealing arrangement 16 is furthermore connected to a headgear 26. The headgear 26 may comprise one or more strap members 28a-28c which may be used for donning the sealing arrangement 16 to the face of the patient 12. The strap members 28a-28c may be guided around the back and/or the top of the skull of the patient 12 in order to secure the patient interface 10 to the head of the patient 12. The strap members 28a-28c are herein also denoted as headgear straps.

One of the central features of the present invention is the fluidic connection between the interior of the sealing arrangement 16 and the interior of the headgear straps 28a-28c. The headgear straps 28a-28c comprise an internal chamber forming a cavity 30 in the interior of the headgear straps 28a-28c. The pressurized flow of breathable gas may thus be guided from the interior of the sealing arrangement 16 into the cavity 30 within the interior of the headgear straps 28a-28c.

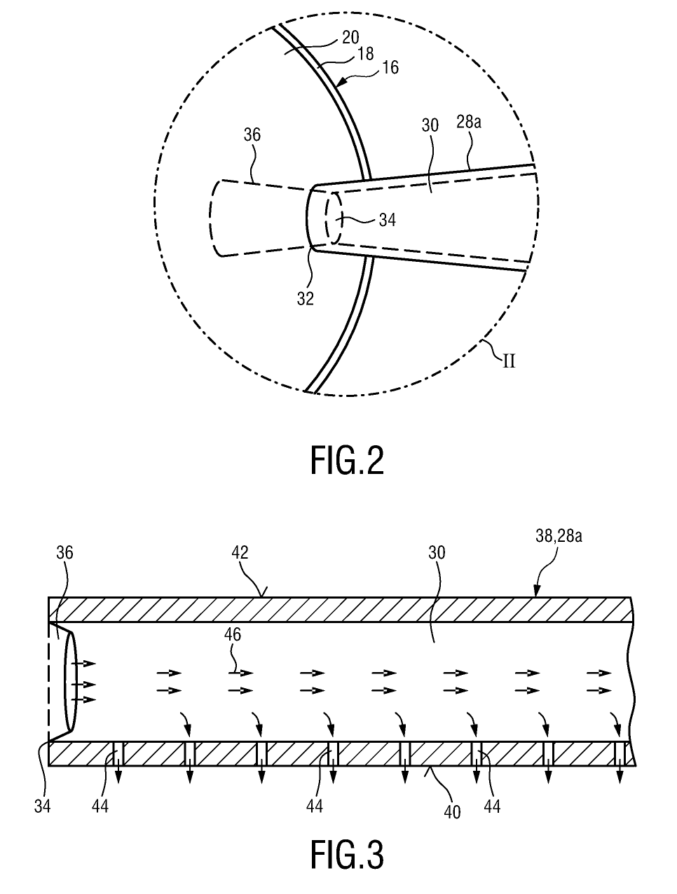

FIG. 2 shows a schematic illustration of the connection between the sealing arrangement 16 and headgear strap 28a. As illustrated therein, the headgear strap 28a comprises a connector 32 for fluidly connecting the cavity 30 to the interior of the sealing arrangement 16. In the shown example the headgear strap 28a is connected to the mask shell 20 by means of the connector 32. FIG. 2 shows one exemplary example of such a connector 32. Therein the connector 32 comprises an inlet opening 34 and a conical tube 36 that is inserted into the inlet opening 34 of the headgear strap 28a. The conical tube 36 is arranged such that the side with the smaller diameter leads into the headgear strap 28a and the side with the larger diameter is facing towards the interior of the sealing arrangement 16. This ensures a mechanically stable connection between the sealing arrangement 16 and the headgear strap 28a, since the conical tube 36 will be automatically pressed into the cavity 30 by means of the pressure occurring within the interior of the sealing arrangement 16. However, it shall be noted that other types of connectors 32 may be used without leaving the scope of the present invention.

FIG. 3 shows a schematic cross-section of a strap portion 38 of the strap member 28a. The first side 40 of the strap portion 38 is configured to contact the face of the patient 12 during use. The opposite second side 42 of the strap portion 38 is facing away from the face of the patient 12 during use and so to say builds the outer side/top side of the headgear strap 28a shown in FIG. 1.

As it may be seen in FIG. 3, the strap portion 38 comprises a plurality of openings 44 which are arranged at the first side 40. These openings 44 are preferably realized as through holes which lead into the internal chamber 30. The internal chamber 30 is thus fluidly connected to the exterior of the headgear strap 28a by means of these openings 44. The flow of breathable gas generated by the pressure generator 14 is therefore not only supplied to the interior of the sealing arrangement 16, but also into the interior 30 of the headgear 26. As indicated by arrows 46 in FIG. 3, at least a part of the flow of breathable gas, which is guided through the headgear 26, leaves the headgear 26 through the openings 44 towards the face of the patient 12. The flow of breathable gas is therefore not only supplied to the airway of the patient 12, but (at least in part) also supplied to the areas of the patient's face that are covered by the headgear 26. In other words, the generated flow of breathable gas is not only used for ventilation purposes, but also for cooling and drying the areas underneath the headgear straps 28a-28c. This helps to reduce or prevent sweat and a red mark formation underneath the headgear straps 28a-28c.

The openings 44 arranged at the first side 40 of the strap portion 38 may, for example, be realized by means of a perforated material. The second side 42 of the strap portion 38 preferably comprises an air-tight material, such that gas is leaving the internal chamber 30 of the strap portion 38 only towards the patient's face, but not towards the top side of the headgear 26.

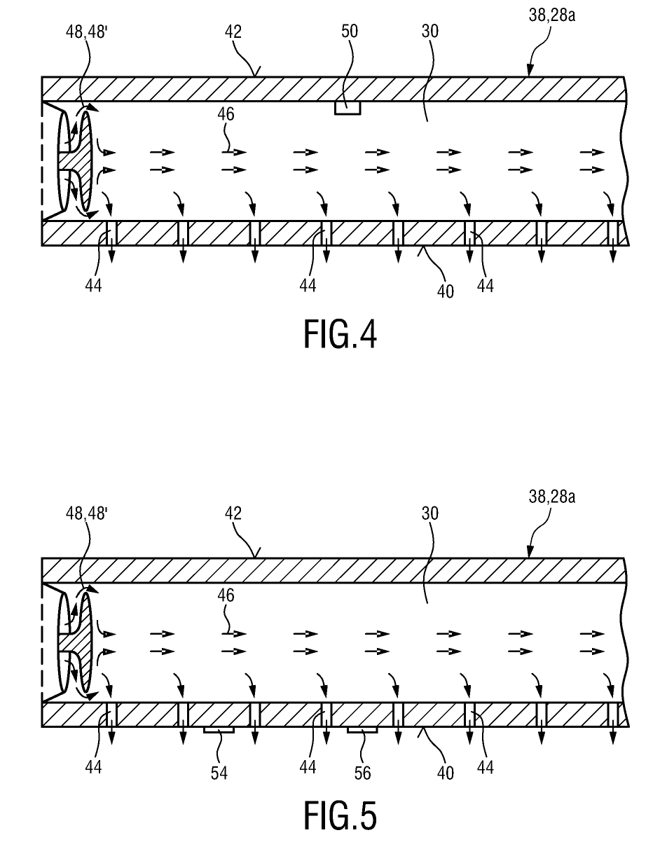

FIG. 4 shows a schematic cross-section of the strap member 28a according to a second embodiment of the present invention. The strap member 28a therein further comprises a flow restriction element 48 for restricting and/or regulating an inflow of the pressurized flow of breathable gas into the cavity 30 of the strap portion 38. This flow restriction element 48 may comprise a valve 48′. The valve 48′ helps to regulate the amount of flow guided through the headgear 26 in order to regulate the pressure occurring within the cavity 30. The pressure within the cavity 30 is preferably controlled to be lower than the pressure occurring within the interior of the sealing arrangement 16.

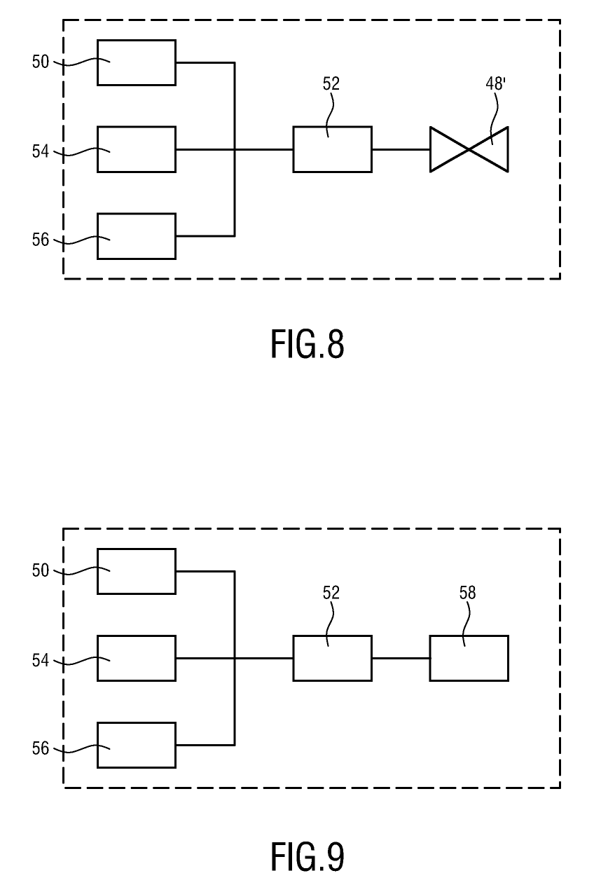

The valve 48′ may be either realized as a mechanical pressure relief valve or as an electronically actuated valve. According to the embodiment shown in FIG. 4 the strap member 28a also comprises a pressure sensor 50 for measuring the pressure within the cavity 30, and a controller 52 (see FIG. 8) for controlling the valve 48′. The controller 52 is configured to control the valve 48′ depending on the pressure measured by the pressure sensor 50. This arrangement may help to regulate the amount of gas entering the cavity 30 and to keep the pressure within the cavity 30 at a fairly constant level.

The controller 52 may, for example, be configured to open the valve 48′ if the pressure measured by the pressure sensor 50 falls below a predefined minimum threshold value, and to close the valve 48′ if the pressure measured by the pressure sensor 50 rises above a predefined maximum threshold value. The pressure sensor 50 and the valve 48′ are both connected to the controller 52 (see FIG. 8). These connections may be either realized as wireless connections or as hard-wired connections. The controller 52 may, for example, be realized as a microprocessor that is either arranged at or within one of the strap members 28a-28c, the sealing arrangement 16 or remote from the patient interface 10.

A further alternative for controlling the valve 48′ is a time-dependent control. The valve 48′ may, for example, be opened during certain predefined time intervals, e.g. every 30 minutes for one minute. The controller 52 may also be configured to open the valve 48′ only during certain times of the day, e.g. only during the night only between 1 a.m. and 5 a.m. when the patient 12 is asleep. This provides the advantage that the patient 12 does not even recognize the cooling and drying action performed by the presented headgear 26.

Instead of a time-dependent control of the valve 48′, the patient interface 10 may further comprise a vital sign sensor (not shown) that is configured to measure a vital sign of the patient, such as a blood pressure, a blood pulse or blood oxygenation. Such a vital sign sensor may, for example, comprise a photoplethysmographic (PPG) sensor. Connecting such a sensor to the controller 52 would allow the detection of a sleep state of the patient 12 based upon the measured vital sign(s). The controller 52 may in this case be configured to control the valve 48′ depending on a sleep state of the patient 12, i.e. open the valve 48′ only if the patient 12 is asleep.

Still further alternatives of controlling the valve 48′, i.e. of controlling the cooling and drying action of the herein presented headgear 26, are schematically shown in FIG. 5. According to the therein shown alternative, the strap member 28a may further comprise a humidity sensor 54 and/or a temperature sensor 56 arranged at the first side 40 of the strap member 28a. These sensors 54, 56 may be also connected to the controller 52 either by means of a wireless connection or by means of a hard-wired connection. The controller 52 may be configured to control the valve 48′ depending on the humidity/temperature measured by the humidity sensor 54/temperature sensor 56. Gas may thus be supplied to the areas underneath the headgear 26 only if a certain temperature or humidity threshold value underneath the headgear 26 is exceeded. The cooling and drying action is thus only provided if really needed. Otherwise, the valve 48′ is closed and the full amount of pressure is supplied to the airway of the patient 12.

It should be clear that the pressure sensor 50, the humidity sensor 54, the temperature sensor 56 and the vital sign sensor may also be used altogether such that the controller 52 controls the valve 48′ based upon the signals of all these sensors.

In the herein shown example, the pressurized flow of breathable gas is provided directly into the interior of the sealing arrangement 16 and only indirectly into the interior of the headgear 26. Alternatively, it is however also possible to connect the pressure generator 14 directly to the interior 30 of the headgear 26, such that the pressurized flow of breathable gas first enters the headgear 26 and is then guided into the interior of the sealing arrangement 16.

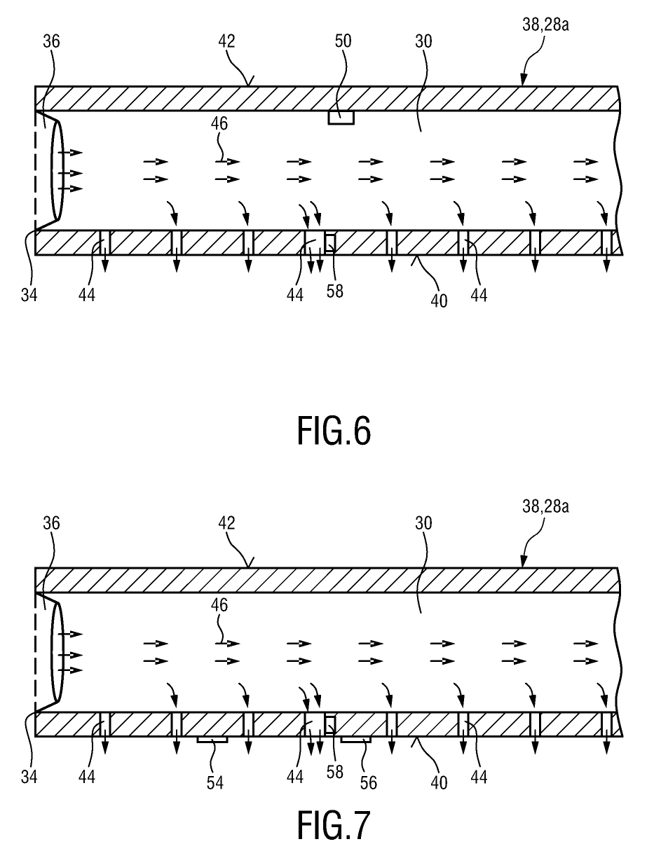

FIG. 6 shows a schematic cross-section of the strap member 28a according to a fourth embodiment of the present invention. The strap member 28a therein further comprises an actuator 58 for regulating an outflow of the pressurized flow of breathable gas through at least one of the plurality of openings 44 out of the cavity 30 by changing at least one of a size, a shape, a location, and a geometry of the at least one of the plurality of openings 44. This actuator 58 helps to regulate the amount of flow guided through the openings 44 in order to cool the skin of the patient 12 underneath the headgear 26. The flow rate of the gas flow for cooling the skin of the patient 12 is preferably controlled to be lower than the flow rate of the gas flow entering the interior of the sealing arrangement 16.

The actuator 58 is preferably realized as an electronically actuated actuator and may e.g. comprise an electromechanical valve. It shall be noted that in FIGS. 6 and 7 only one actuator 58 is shown. Alternatively, the strap member 28a may comprise a plurality of such actuators 58, one for each opening 44. According to a further alternative, only one common actuator 58 may be configured to change a size, a shape, a location, and a geometry of the plurality of openings 44.

According to the embodiment shown in FIG. 6 the strap member 28a also comprises a pressure sensor 50 for measuring the pressure within the cavity 30, and a controller 52 (see FIG. 9) for controlling the actuator 58. The controller 52 is configured to control the actuator 58 depending on the pressure measured by the pressure sensor 50. This arrangement may help to regulate the amount of gas leaving the cavity 30 through the openings 44.

The controller 52 may, for example, be configured to control the actuator 58 so as to enlarge the cross-section of the corresponding opening 44 if the pressure measured by the pressure sensor 50 falls below a predefined minimum threshold value, and to reduce the size of the cross-section of the corresponding opening 44 if the pressure measured by the pressure sensor 50 rises above a predefined maximum threshold value. The pressure sensor 50 and the actuator 58 are both connected to the controller 52 (see FIG. 9). These connections may be either realized as wireless connections or as hard-wired connections.

A further alternative for controlling the actuator 58 is a time-dependent control. The opening(s) 44 may, for example, be opened by the actuator 58 during certain predefined time intervals, e.g. every 30 minutes for one minute. The controller 52 may also be configured to control the actuator 58 to open the opening(s) 44 only during certain times of the day, e.g. only during the night only between 1 a.m. and 5 a.m. when the patient 12 is asleep. This provides the advantage that the patient 12 does not even recognize the cooling and drying action performed by the presented headgear 26.

The actuator 58 may, similar as explained above for the valve 48′, also be controlled based on signals of a vital sign of the patient, such as a blood pressure, a blood pulse or blood oxygenation. Connecting such a sensor to the controller 52 would allow the detection of a sleep state of the patient 12 based upon the measured vital sign(s). The controller 52 may in this case be configured to control the actuator 58 depending on a sleep state of the patient 12.

FIG. 7 shows a schematic cross-section of the strap member 28a according to a fourth embodiment of the present invention. According to the therein shown alternative, the strap member 28a may further comprise a humidity sensor 54 and/or a temperature sensor 56 arranged at the first side 40 of the strap member 28a. These sensors 54, 56 may be also connected to the controller 52 either by means of a wireless connection or by means of a hard-wired connection. The controller 52 may be configured to control the actuator 58 depending on the humidity/temperature measured by the humidity sensor 54/temperature sensor 56. Gas may thus be supplied to the areas underneath the headgear 26 only if a certain temperature or humidity threshold value underneath the headgear 26 is exceeded. The cooling and drying action is thus only provided if really needed.

It should be clear that the pressure sensor 50, the humidity sensor 54, the temperature sensor 56 and the vital sign sensor may also be used altogether such that the controller 52 controls the actuator 58 based upon the signals of all these sensors. It shall be also clear that the embodiments including the valve 48′ (see FIGS. 4 and 5) may be combined with the embodiments including the actuator 58 (see FIGS. 6 and 7).

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A strap member for use in a patient interface for supplying a pressurized flow of breathable gas to an airway of a patient, wherein the strap member is configured to secure the patient interface to the head of the patient and comprises:
a strap portion having (i) a first side that is configured to contact the face of the patient during use, (ii) an internal chamber forming a cavity, and (iii) a plurality of openings (44) which are fluidly connected to the cavity and arranged at the first side;
a flow restriction element for restricting and/or regulating an inflow of the pressurized flow of breathable gas into the cavity; and
a connector for fluidly connecting the cavity to the pressurized flow of breathable gas supplied to the airway of the patient during use of the patient interface.

2. The strap member according to claim 1, wherein a second side of the strap portion opposite the first side comprises a substantially air-tight material.

3. The strap member according to claim 1, wherein the first side of the strap portion comprises a perforated material which comprises the plurality of openings.

4. The strap member according to claim 1, wherein the flow restriction element comprises a valve.

5. The strap member according to claim 4, wherein the valve is arranged at or near the connector.

6. The strap member according to claim 4, further comprising a pressure sensor for measuring a pressure within the cavity, and a controller for controlling the valve, wherein the controller is configured to control the valve depending on the pressure measured by the pressure sensor.

7. The strap member according to claim 4, further comprising a controller for controlling the valve, wherein the controller is configured to open the valve during predetermined time intervals.

8. The strap member according to claim 4, further comprising a humidity sensor for sensing a humidity at or near the first side, of the strap portion, and a controller for controlling the valve, wherein the controller is configured to control the valve depending on the humidity measured by the humidity sensor.

9. The strap member according to claim 4, further comprising a temperature sensor for sensing a temperature at or near the first side of the strap portion, and a controller for controlling the valve, wherein the controller is configured to control the valve depending on the temperature measured by the temperature sensor.

10. The strap member according to claim 1, wherein the connector comprises an inlet opening and a conical tube that is inserted into the inlet opening.

11. A patient interface for supplying a pressurized flow of breathable gas to an airway of a patient, comprising:
a sealing arrangement for providing a substantially air-tight sealing connection with an airway opening of the patient;
an inlet for receiving the pressurized flow of breathable gas which is fluidly connected to an interior of the sealing arrangement; and
a headgear including a strap member as claimed in claim 1;
wherein the interior of the sealing arrangement is fluidly connected to the cavity of the strap member by means of the connector.

12. The patient interface according to claim 11, wherein the inlet is arranged at the sealing arrangement.

13. The patient interface according to claim 11, wherein the inlet is arranged at the strap member.

14. A pressure support system comprising:
a pressure generator for generating a pressurized flow of breathable gas; and
a patient interface according to claim 11.

15. A strap member for use in a patient interface for supplying a pressurized flow of breathable gas to an airway of a patient, wherein the strap member is configured to secure the patient interface to the head of the patient and comprises:
a strap portion having (i) a first side that is configured to contact the face of the patient during use, (ii) an internal chamber forming a cavity, and (iii) a plurality of openings which are fluidly connected to the cavity and arranged at the first side;
an actuator for regulating an outflow of the pressurized flow of breathable gas through at least one of the plurality of openings out of the cavity by changing at least one of a size, a shape, a location, and a geometry of the at least one of the plurality of openings; and
a connector for fluidly connecting the cavity to the pressurized flow of breathable gas supplied to the airway of the patient during use of the patient interface.

16. The strap member according to claim 15, wherein the actuator is arranged at or near the at least one of the plurality of openings.

17. The strap member according to claim 15, further comprising a pressure sensor for measuring a pressure within the cavity a, and a controller for controlling the actuator wherein the controller is configured to control the actuator depending on the pressure measured by the pressure sensor.

18. The strap member according to claim 15, further comprising a controller for controlling the actuator, wherein the controller is configured to control the actuator to change the outflow of the pressurized flow of breathable gas through the at least one of the plurality of openings during predetermined time intervals.

19. The strap member according to claim 15, further comprising a humidity sensor for sensing a humidity at or near the first side of the strap portion, and a controller for controlling the actuator, wherein the controller is configured to control the actuator to change the outflow of the pressurized flow of breathable gas through the at least one of the plurality of openings depending on the humidity measured by the humidity sensor.

20. The strap member according to claim 15, further comprising a temperature sensor for sensing a temperature at or near the first side of the strap portion, and a controller for controlling the actuator, wherein the controller is configured to control the actuator to change the outflow of the pressurized flow of breathable gas through the at least one of the plurality of openings depending on the temperature measured by the temperature sensor.
